(19) United States
(12) Patent Application Publication
(10) Pub. No.: US 2006/0198809 A1
(43) Pub. Date: Sep. 7, 2006

(54) Gamma-amino butyric acid composition

(75) Inventor: Erica Herrel, Newbury Park, CA (US)

Correspondence Address: JEFFER, MANGELS, BUTLER & MARMARO, LLP, 1900 AVENUE OF THE STARS, 7TH FLOOR, LOS ANGELES, CA, 90067, US

(73) Assignee: Woodridge Labs, Inc., Van Nuys, CA (US)

(21) Appl. No.: 11/072,184

(22) Filed: Mar. 4, 2005

(51) Int. Cl. A61K 8/97; A61K 36/254; A61K 36/48; A61K 36/258

(52) U.S. Cl. 424/74; 424/757; 424/725; 424/728

(57) ABSTRACT

A composition for reducing skin wrinkles comprising a carrier and at least two neuromascular inhibitors. The composition can further include botanicals to further facilitate the reduction of fine lines, as well as emollients, thickeners and humectants.

### FIELD OF THE INVENTION

The present invention relates generally to a composition for reducing wrinkles in skin. More particularly, the invention relates to a composition that contains gamma-amino butyric acid and helps reduce wrinkles in the skin.

### BACKGROUND OF THE INVENTION

Women and men, today seek to maintain a youthful appearance for as long as possible and, consequently, seek to attenuate the signs of skin aging, which manifest themselves especially by normal and small wrinkles. In this regard, advertisements and fashion promote products intended to maintain a radiant skin for as long as possible and without wrinkles, which is the sign of a youthful skin, all the more so since physical appearance is important for peace of mind and/or for morale. Thus, it is important to feel physically young.

To date, normal and small wrinkles were treated with cosmetic products containing active agents acting on the skin, for example by moisturizing it or by enhancing its cellular renewal or by promoting the synthesis of collagen which constitutes the cutaneous tissue. However, also to date, it was not known to this art how to treat wrinkles by acting on the muscular elements present in the skin.

It is known that the facial platysma muscles are under the control of the motor nerve afferences of the facial nerve and that, moreover, the interlobular septa of the hypodermis contain therein fibers which constitute a striated muscular tissue (panniculus carnosus). Too, it is also known that a subpopulation of fibroblasts of the dermis, designated myofibroblasts, exhibits characteristics which are common with the muscular tissue.

In certain pathological and therapeutic conditions, the influence exerted on facial wrinkles by the nerves controlling all of this muscular tissue has been observed. Thus, in facial nerve conditions, in which the transmission of the nerve impulse is. interrupted and/or reduced, there is observed in the area of innervation a paralysis of the facial muscles. This facial paralysis results, besides other clinical signs, in an attenuation, or even disappearance of the wrinkles.

In contrast, in the states of facial muscular hypercontraction, accentuation of facial wrinkles has also been observed. Furthermore, an accentuation of the facial wrinkles in the muscular hypertonia states in Parkinson's disease and side effects induced by neuroleptics too has been observed.

Moreover, it has been demonstrated that the botulinus toxin, originally used for treating spasms, is active on muscular spasticity states and on the wrinkles on the glabella which are intersuperciliary wrinkles. Consequently, it is possible to influence, via a pharmacological action, the nervous component of the wrinkles.

In the peripheral nervous system, the junction between a nerve and a muscle constitutes the neuromuscular plate, upstream of which there is the afferent nerve route denominated motoneuron. Moreover, the cellular membranes of each nerve fiber comprise numerous ion channels, and especially chlorine channels, capable of permitting the corresponding element in ionic form, and in the case of the chlorine channels in chloride form, to pass therethrough. These channels are associated with neuronal receptors. The neuronal receptors associated at the periphery with the chlorine channels are especially receptors for glycine (glycine-strychnine sensitive receptors) and receptors for type gamma-amino butyric acid (hereinafter “GABA”).

It too is known that it is possible to reduce the excitability of the motoneuron by various pharmacological agents acting on the glycine-strychnine sensitive receptors or on the GABA-A receptors of the peripheral nervous system. Thus, it is possible to modulate the excitability of the motoneuron, for example by GABA.

The activation of these receptors opens the chlorine channels and permits entry of chloride ions, which results in an increase in the chloride ions in the cells of the nerve fiber and therefore to a hyperpolarization of the motoneurons which become, as a result, less excitable. This reduction in excitability of the motoneuron causes a lesser stimulation of the muscle fiber, thereby effecting its loosening.

### SUMMARY OF THE PREFERRED EMBODIMENTS

In accordance with one aspect of the present invention, there is provided a composition for reducing the appearance of wrinkles comprising at least two neuromascular inhibitors, at least one botanical and a carrier.

Other objects, features and advantages of the present invention will become apparent to those skilled in the art from the following detailed description. It is to be understood, however, that the detailed description and specific examples, while indicating preferred embodiments of the present invention, are given by way of illustration and not limitation. Many changes and modifications within the scope of the present invention may be made without departing from the spirit thereof, and the invention includes all such modifications.

### DETAILED OF THE PREFERRED EMBODIMENTS

The present invention provides a composition containing GABA for inhibiting neuromuscular impulses, effectively, numbing the muscles to relax fine lines and wrinkles. The compositions for topical application may advantageously constitute a cosmetic or pharmaceutical composition for protection, treatment or care of the face, the neck, the hands or the body, (for example day creams, night creams, sunscreen creams or oils, lotions, body milks), a makeup composition (for example foundation) or a composition for artificial tanning.

For topical application, the compositions of the invention comprise a medium which is compatible with water. These compositions may be provided, especially, in the form of aqueous, alcoholic or aqueous/alcoholic solutions, of gels, of lotions, of ointments, of water-in-oil or oil-in-water emulsions having the appearance of a cream or of a gel, of microemulsions, of aerosols, or in the form of vesicular dispersions containing ionic and/or nonionic lipids.

The compositions according to the invention may be administered either locally, namely, topically, or by subcutaneous and/or intradermal injection, or systemically or generally, i.e., orally and/or by intramuscular injection.

A composition according to a preferred embodiment of the present invention is set forth according to the table below, with the percentages by weight.

| | | Percent | Preferred |
|---|---|---|---|
| Ingredient | Function | Range | Range |
| deionized water | carrier | 50-100% | 50-70% |
| glyceryl stearate | emulsifier | 1-10% | 3-8% |
| sesame oil | emollient | 1-10% | 2-6% |
| caprylie/capric | emollient | 1-10% | 2-5% |
| triglyceride | | | |
| PEG-150 | thickener | 1-6% | 3-5% |
| Distearate | | | |
| glycyrrhiza | neuromascular | 0.01-5% | 1-5% |
| glabra | inhibitor | | |
| cetyl alcohol | thickener | 1-5% | 1-3% |
| cetearyl alcohol | thickener | 1-5% | 1-3% |
| butylene glycol | humectant | 1-5% | 1-3% |
| panax ginseng | botanical | 0.001-1% | 0.001-1% |
| gamma | neuromascular | 0.001-0.5% | 0.001-0.05% |
| aminobutyric | inhibitor | | |
| acid | | | |
| ethoxydiglycol | solvant | 0.1-10% | 0.5-5% |
| xanthan gum | thickener | 0.01-1% | 0.01-0.5% |
| eugenol | botanical | 0.01-0.5% | 0.05-0.5% |
| gynostemma | botanical | 0.0001-1% | 0.0001-0.1% |
| pentaphyllum | | | |
| orchid extract | botanical | 0.0001-1% | 0.0001-0.1% |
| propylene glycol | humectant | 0.1-1% | 0.1-1% |
| methylparaben | preservative | 0.05-0.5% | 0.05-0.2% |
| propylparaben | preservative | 0.01-0.3% | 0.01-0.2% |
| diazolidinyl urea | preservative | 0.05-0.5% | 0.05-0.5% |
| bismuth | light | 0.01-2.0% | 0.01-1% |
| oxychloride | diffusing | | |
| | pigment | | |

One other preferred “active” ingredient in the inventive formula is the glycyrrihza glabra. In a most preferred embodiment it is present in the formula at 3%.

The ethoxydiglycol is a transdermal delivery solvent that is most effective at levels higher than 0.5%. It works in combination with the active ingredient in inhibiting neuromuscular impulses, effectively, numbing the muscles to relax fine lines and wrinkles.

The combination of the following preferred ingredients work together to reduce the appearance of fine lines and wrinkles: glycyrrhiza glabra, ginseng extract, gama aminobutyric acid, gynostemma pentaphyllum, orchid extract and ethoxydiglycol.

Bismuth oxychloride is a pigment that is added for light diffusing. This ingredient comes in different forms, however the INCI name is always bismuth oxychloride. Accordingly, those skilled in the art will be aware of suitable substitutes. Such substitutes are within the scope of the invention.

Sesame oil is a natural oil used as a skin conditioner. Any other natural oil will have the same function. Accordingly, all naturally derived oils can be used. For example, safflower oil or sunflower oil can be used as substitutes.

Glyceryl stearate is a common glyceride emulsifier. Cetyl alcohol can be substituted with stearyl alcohol or any other known fatty alcohol.

In known fashion, the cosmetic or pharmaceutical compositions of the invention may also contain customary additives and adjuvants in the corresponding fields, such as hydrophilic or lipophilic gelling agents, preservatives, antioxidants, solvents, perfumes, fillers, UV-screening agents and colorants. Moreover, these compositions may contain hydrophilic or lipophilic active agents. The amounts of these different additives and adjuvants or active agents are those conventionally used in the cosmetic or pharmaceutical field, and range, for example, from 0.01% to 20% of the total weight of the composition. These adjuvants/additives or these active agents, depending on their particular nature, may be introduced in the fatty phase, in the aqueous phase and/or in the lipid vesicles.

In a preferred embodiment, a cream is made by heating the water to 80° C. and adding the butylene glycol and xanthan gum (with fast mixing). The oils and waxes are heated to melt in a separate vessel. Bismuth oxychloride is added to the oil phase and blended until no lumps are visible. The melted and blended waxes and oils are than added to hot water. The formula is mixed for 20 minutes, then cooled to room temperature.

The botanicals and active ingredients are blended together with ethoxydiglycol and are added once the formulation is at room temperature. Any extraction technique known to the art may be used to prepare the botanical extracts contained in the composition according to the invention. At this time, the preservatives are added as well.

In order to further illustrate the present invention and the advantages thereof, the following chart gives a specific example of a composition according to the present invention, it being understood that it is intended only as illustrative and in no way limitative.

| Ingredient | Weight Percent |
|---|---|
| Water | to 100.0% |
| butylene glycol | 1.000 |
| Xanthan gum | 0.25 |
| glyceryl stearate | 7.00 |
| sesame oil | 5.00 |
| caprylic/caprie triglyceride | 4.00 |
| PEG-150 distearate | 3.00 |
| cetyl alcohol | 2.00 |
| cetearyl alcohol | 2.00 |
| Bismuth oxychloride | 0.25 |
| Ginseng extract | 1.00 |
| gams aminobutyric acid | 0.02 |
| ethoxydiglycol | 0.50 |
| orchid extract | 0.01 |
| gynostemma pentaphyllum | 0.01 |
| glycyrrhiza glabra | 3.00 |
| propylene glycol | 0.70 |
| methylparaben | 0.20 |
| propylparaben | 0.05 |
| diazolidinyl urea | 0.20 |
| Eugenol | 0.05 |

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. A composition for reducing the appearance of wrinkles comprising at least two neuromascular inhibitors, at least one botanical and a carrier.

2. The composition of claim 1 further including a humectant.

3. The composition of claim 2 further including a thickener.

4. The composition of claim 1 wherein there are at least three botanicals.

5. The composition of claim 1 wherever the neuromascular inhibitors are glycyrrhiza glabra and gamma aminobutyric acid.

6. A composition containing at least 50% deionized water; at least 1% glyceryl stearate; at least 1% sesame oil; at least 1% caprylie/capric triglyceride; at least 1% PEG-150 Distearate; at least 0.01% glycyrrhiza glabra; at least 1% cetyl alcohol; at least 1% cetearyl alcohol; at least 1% butylene glycol; at least 0.001% panax ginseng; at least 0.001% gamma aminobutyric acid; at least 0.1% ethoxydiglycol; at least 0.01% xanthan gum; at least 0.01% eugenol; at least 0.0001% gynostemma pentaphyllum; at least 0.0001% orchid extract; at least 0.1% propylene glycol; at least 0.05% methylparaben; at least 0.01% propylparaben; at least 0.05% diazolidinyl urea; and at least 0.01% bismuth oxychloride.

7. A composition containing water; 1% butylene glycol; 0.25% Xanthan gum; 7% glyceryl stearate; 5% sesame oil; 4% caprylic/caprie triglyceride; 3% PEG-150 distearate; 2% cetyl alcohol; 2% cetearyl alcohol; 0.25% Bismuth oxychloride; 1% Ginseng extract; 0.02% gams aminobutyric acid; 0.5% ethoxydiglycol; 0.01% orchid extract; 0.01% gynostemma pentaphyllum; 3% glycyrrhiza glabra; 0.7% propylene glycol; 0.2% methylparaben; 0.05% propylparaben; 0.2% diazolidinyl urea; and 0.05% Eugenol.

8. A composition comprising about 0.01 to 5% of a first neuromascular inhibitor, about 0.1 to 10% carrier, about 0.001 to 1% of a first botanical, about 0.001 to 0.5% of a second neuromascular inhibitor, about 0.0001 to 1% of a second botanical, and about 0.0001 to 1% of a third botanical.

9. The composition according to claim 1 comprising about 1 to 5% of a first neuromascular inhibitor, about 0.5 to 5% solvant, about 0.001 to 1% of a first botanical, about 0.001 to 0.05% of a second neuromascular inhibitor, about 0.0001 to 0.1% of a second botanical, and about 0.0001 to 0.1% of a third botanical.

10. The composition according to claim 1 wherein the first neuromascular inhibitor is glycyrrihza glabra, the first botanical is panax ginseng, the second neuromascular inhibitor is gama aminobutyric acid, the second botanical is gynostemma pentaphyllum, and the third botanical is orchid extract.

11. The composition according to claim 2 wherein the first neuromascular inhibitor is glycyrrihza glabra, the first botanical is panax ginseng, the second neuromascular inhibitor is gama aminobutyric acid, the second botanical is gynostemma pentaphyllum, and the third botanical is orchid extract.
